(19) United States
(12) Patent Application Publication
(10) Pub. No.: US 2024/0350030 A1
(43) Pub. Date: Oct. 24, 2024

(54) ELECTRONIC RULER FOR PETS, BODY INDEX MEASUREMENT SYSTEM, AND PET ACCESSORIES RECOMMENDATION SYSTEM

(71) Applicant: Guilin Gemred Sensor Technology Co., Ltd., Guilin (CN)

(72) Inventors: Wenjing HE, Guilin (CN); Guanglu YANG, Guilin (CN); Ye CHEN, Guilin (CN); Mengying SHI, Guilin (CN); Jinhua LAN, Guilin (CN); Jiangwei LU, Guilin (CN)

(21) Appl. No.: 18/211,232

(22) Filed: Jun. 16, 2023

(30) Foreign Application Priority Data
Apr. 18, 2023 (CN) 202310416675.3

(51) Int. Cl. A61B 5/107; A61B 5/00

(52) U.S. Cl. CPC A61B 5/1072; A61B 5/0022; A61B 5/1079; A61B 2503/40

(57) ABSTRACT

An electronic ruler for pets includes a displacement sensor module; a controller; a display; a first wireless communication module; and a control key. The displacement sensor module includes a tape or reel; the tape or reel is configured to convert a physical length of a pet to be measured into a displacement length through a rotation angle of the tape or reel, and the displacement length is further converted into an electrical signal; the controller is configured to calculate and convert the electrical signal into a length value in a set unit, and display the length value on the display; and the first wireless communication module is configured to transmit the length value to an upper computer, and the control key is configured to set the parameters of the length value and confirm the transmission of the length value to the to the upper computer.

### CROSS-REFERENCE TO RELATED APPLICATIONS

Pursuant to 35 U.S.C. § 119 and the Paris Convention Treaty, this application claims foreign priority to Chinese Patent Application No. 202310416675.3 filed Apr. 18, 2023, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to the field of electronic rulers and pet body index measurement, and more particularly to an electronic ruler for pets, a pet body index measurement system and a pet accessories recommendation system.

At present, conventional soft rulers are used to measure the body circumference of pets. Such soft rulers require manual measurement and manual reading. Therefore, such soft rulers have the following disadvantages.

1. Easily lead to errors or even mistakes in reading due to human subjective reasons.

2. The data needs to be manually recorded during measurement, so it is inconvenient to save and process the measured data, and it is also impossible to visually present the circumference data of the pet in a period of time.

3. The function cannot be extended. During the measurement of the body circumference of the pet, obtaining the circumference data is only the most basic goal of the measurement behavior, and the core goal is to master the health state of the pet and provide guidelines and suggestions for feeding behaviors. It is difficult to satisfy this requirement by using inconvenient soft rulers.

For general electronic rulers, the application scenarios and actual characteristics of measuring pets are not taken into consideration. In this application scenario, at least two factors need to be taken into consideration:

1. The pet may not cooperate completely during measurement. Therefore, the artificial measurement error may be further serious on the basis of the original error.

2. Since different kinds of pets are very different in body shape and there may be cognitive differences or errors for the specific measurement part, it is likely to result in the inconsistency between the measurement result and the actual data and the misjudgment of the body shape and body index data of pets.

### SUMMARY

In view of the above problems, the disclosure discloses an electronic ruler for pets, which overcomes the disadvantages of measurement using conventional soft rulers, improves the design of general electronic rulers and solves the problems of electronic rulers when measuring the circumference of pets.

An electronic ruler for pets is provided, comprising a displacement sensor module; a controller; a first display; a first wireless communication module; and a control key. The displacement sensor module comprises a tape or reel; the tape or reel is configured to convert a physical length of a pet to be measured into a displacement length through a rotation angle of the tape or reel, and the displacement length is further converted into an electrical signal; the controller is configured to calculate and convert the electrical signal into a length value in a set unit, and display the length value on the first display; and the first wireless communication module is configured to transmit the length value to an upper computer, and the control key is configured to set the parameters of the length value and confirm the transmission of the length value to the to the upper computer.

In a class of this embodiment, to realize high-accuracy and high-speed measurement, the displacement sensor module is an absolute capacitive grating sensor or an optical grating sensor.

In a class of this embodiment, the first wireless communication module is a Bluetooth module, a WIFI module, a ZigBee module, an NFC module and/or an Ethernet module.

In a class of this embodiment, to set the parameters of the length value comprises one or more of the following: setting the unit of the length value, setting or selecting the position where the length value belongs to the pet, selecting to delete or reserve the length value, selecting the average/sum/difference of several length values, and setting or selecting the length value as an absolute length or a relative length.

In accordance with another aspect of the disclosure, a pet body index measurement system is provided, comprising the electronic ruler for pets and an upper computer, wherein the upper computer comprises a memory and a second display; the memory is configured to save the pet body index data; the second display is configured to display the pet body index data and exhibit a setup interface for interaction with the electronic ruler for pets; the upper computer is configured to calculate and convert the pet body index data and perform wireless communication with the electronic ruler for pets through the first wireless communication module; and, the electronic ruler for pets digitally acquires the length data of the body part of the pet, and the first wireless communication module is configured to transmit the length data and receive a control signal transmitted by the upper computer.

In a class of this embodiment, the measurement system further comprises an electronic scale comprising a second wireless communication module; and the upper computer is configured to calculate and convert the pet body index data, perform wireless communication with the electronic ruler through the first wireless communication module and/or perform wireless communication with the electronic ruler through the second wireless communication module.

The electronic ruler digitally acquires the length data of the body part of the pet, and the first wireless communication module is configured to transmit the length data and receive the control signal transmitted by the upper computer; the electronic scale digitally acquires the weight data of the pet, and the second wireless communication module is configured to transmit the weight data and receive the control signal transmitted by the upper computer; and, the upper computer calculates BMI and BCS scores for the pet according to the variety of the pet, the length data and the weight data.

In a class of this embodiment, the electronic rule comprises keys for numbering and confirming the measured length data before the first wireless communication module transmits the length data.

In a class of this embodiment, the length data comprises one or more length data of neck circumference, chest circumference, body length, body height, leg length, tail length, claw width, mouth circumference, and leg circumference.

In a class of this embodiment, the information received by the first wireless communication module comprises the measurement time of the length data, and the information received by the second wireless communication module comprises the measurement time of the weight data; the length data displayed on the display comprises: under a length index, the horizontal axis is a set time range interval, and the vertical axis is a connection graph of the length data in the time range interval; and, the weight data displayed on the display comprises: under a weight index, the horizontal axis is a set time range interval, and the vertical axis is a connection graph of the weight data in the time range interval, or a length and/or weight display graph of the pet at a single specified time from different perspectives.

In a class of this embodiment, the control signal comprises setting the numbering of the measured length data, the unit of the length data and the transmission mode.

In a class of this embodiment, the upper computer is an intelligent terminal device, a server or a PC computer.

In a class of this embodiment, the upper computer comprises an interface for selecting the variety and/or type of the pet, or a camera for shooting a picture of the pet and an algorithm unit for identifying the variety and/or type of the pet according to the picture of the pet.

In accordance with another aspect of the disclosure, a pet accessories recommendation system is provided, comprising the pet body index measurement system, wherein the recommendation system is configured to complete one or more of the following pet accessories recommendations according to the length data and/or the weight data: calculating the space size of an outer box required by the pet, calculating the size of the pet's clothes or shoes and socks, and calculating the caloric value and/or food intake and/or amount of exercise required by the pet per day.

In accordance with the electronic ruler for pets disclosed by the disclosure, manual reading is not needed, thus avoiding reading errors and realizing high measurement speed; and, the measured data also does not need to be recorded manually, thus realizing high measurement efficiency. The measurement results can be deleted, saved, averaged and/or summated/differentiated during the measurement process, so that the measured data can be flexibly selected as an absolute length or relative length. The accuracy of the measured data is effectively ensured when the pet does not cooperate. The wireless communication with the upper computer is realized through the first wireless communication module, so that it is convenient to transmit, save, display and process the measured data.

In accordance with the pet body index measurement system and the pet accessories recommendation system disclosed by the disclosure, in combination with the upper computer and the electronic scale, the visual displaying of the results of the body index of the pet and the BMI and BCS scores for the pet and the recording, analysis and application of the data are realized. For example, the variation trend in a period of time provides guidelines and suggestions for the purchase of matched surrounding goods and the feeding of the pet.

### BRIEF DESCRIPTION OF THE DRAWINGS

FIG. 1 is a side sectional view of the electronic ruler for pets;

FIG. 2 is a front view of the electronic ruler for pets;

FIG. 3A and FIG. 3B are a left side view and a right side view of the electronic ruler for pets, respectively;

FIG. 4 is a front view of the electronic ruler for pets after the front cover and the display are removed;

FIG. 5 is a front view of the electronic ruler for pets after the front cover/display and the controller are removed;

FIG. 6 is a schematic diagram of connecting the electronic ruler for pets to an intelligent terminal device through Bluetooth;

FIG. 7 is a schematic diagram of connecting the electronic ruler for pets and the electronic scale in the pet body index measurement system to an intelligent terminal device through Bluetooth;

FIGS. 8A-8B are schematic diagrams of data synchronization of the electronic ruler for pets and the intelligent terminal device during neck circumference measurement;

FIGS. 9A-9B are schematic diagrams of data confirmation of the electronic ruler for pets and the intelligent terminal device during neck circumference measurement;

FIGS. 10A-10B are schematic diagrams of data synchronization of the electronic ruler for pets and the intelligent terminal device during chest circumference measurement;

FIGS. 11A-11B are schematic diagrams of data confirmation of the electronic ruler for pets and the intelligent terminal device during chest circumference measurement;

FIGS. 12A-12B are schematic diagrams of data synchronization of the electronic ruler for pets and the intelligent terminal device during body length measurement;

FIGS. 13A-13B are schematic diagrams of data confirmation of the electronic ruler for pets and the intelligent terminal device during body length measurement;

FIGS. 14A-14B are schematic diagrams of data synchronization of the electronic ruler for pets and the intelligent terminal device during body height measurement;

FIGS. 15A-15B are schematic diagrams of data confirmation of the electronic ruler for pets and the intelligent terminal device during body height measurement;

FIG. 16 is a schematic diagram of an interface for data storage in the pet body index measurement system;

FIG. 17 is a schematic diagram showing the circumference data of the pet in a period of time;

FIGS. 18A-18B are schematic diagrams of the result of body shape analysis of the pet; and

FIGS. 19A-19B are schematic diagrams of recommendations for the space size of the pet box and the cloth size.

### DETAILED DESCRIPTION

To further illustrate the disclosure, embodiments detailing an electronic ruler for pets, a pet body index measurement system, and a pet accessories recommendation system are described below. It should be noted that the following embodiments are intended to describe and not to limit the disclosure.

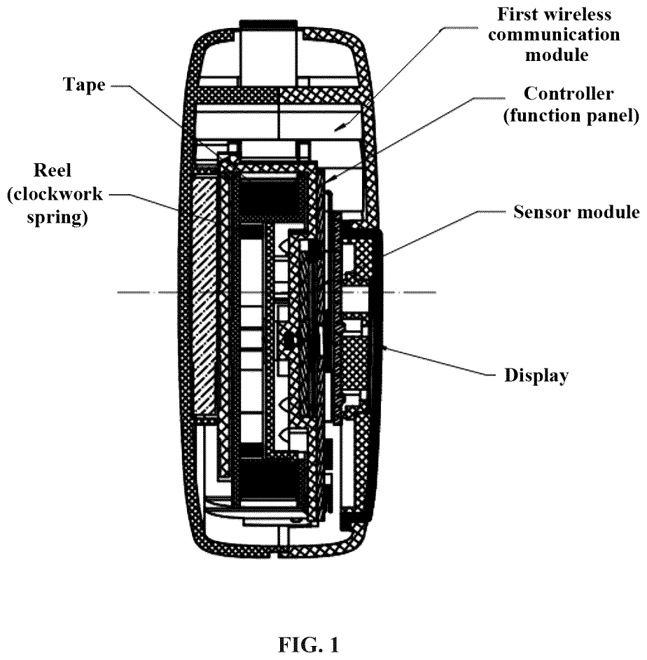

FIG. 1 shows a side sectional view of the electronic ruler for pets. The electronic ruler for pets comprises a displacement sensor module, a controller and a display. The displacement sensor module comprises a tape or reel. The physical length of a pet to be measured is converted into a displacement length through the rotation angle of the tape or reel, and the displacement length is converted into an electrical signal. In order to achieve high measurement accuracy even when the tape or reel is pulled quickly, the displacement sensor module is an absolute capacitive grating sensor or an optical grating sensor. The controller is configured to calculate and convert the electrical signal into a length value in a set unit, and display the length value on the display. The controller can also perform addition, subtraction, multiplication and division on the measurement result. The electronic ruler for pets further comprises a first wireless communication module for sending the length value to an upper computer, and control keys for setting the parameters of the length value and confirming the sending of the length value. The first wireless communication module is connected to an IO port of the controller, to realize data transmission and control command reception/transmission with the upper computer.

Setting the parameter of the length value comprises one or more of the following: setting the unit of the length value, setting or selecting the position where the length value belongs to the pet, selecting to delete or reserve the length value, selecting the average/sum/difference of several length values, and setting or selecting the length value as an absolute length or a relative length.

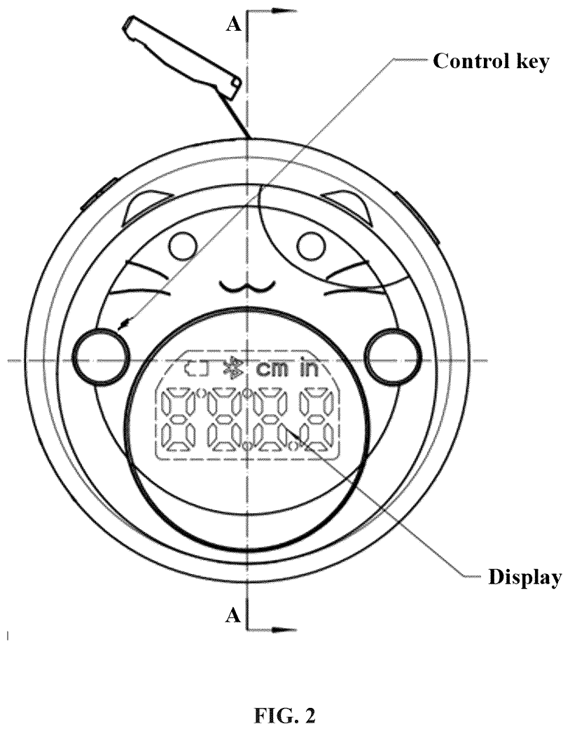

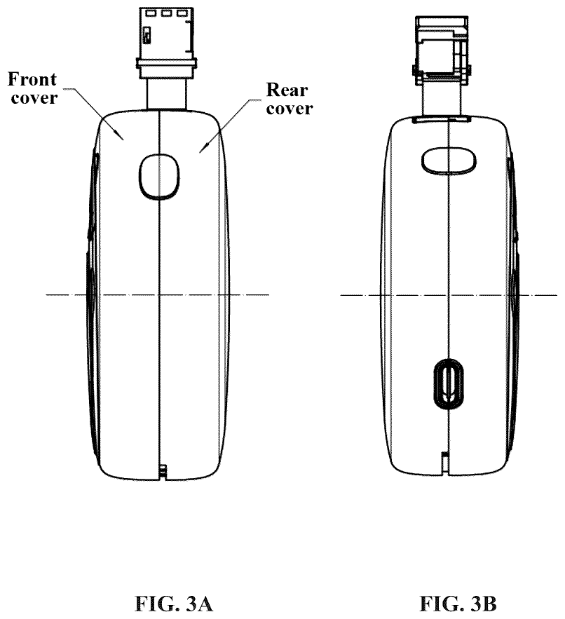

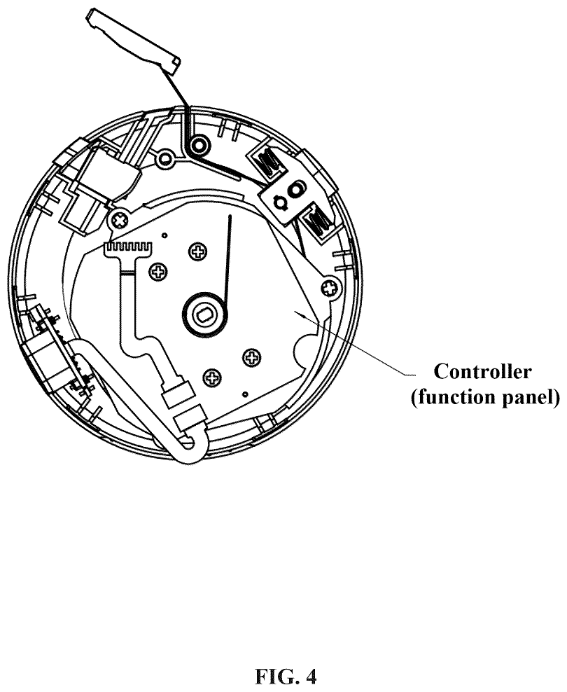

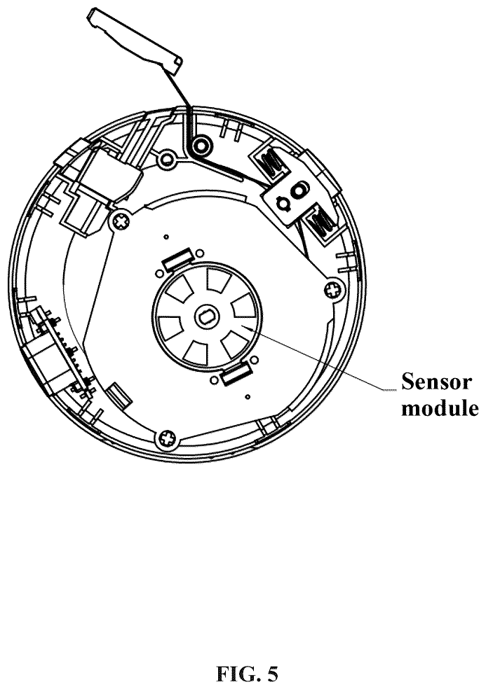

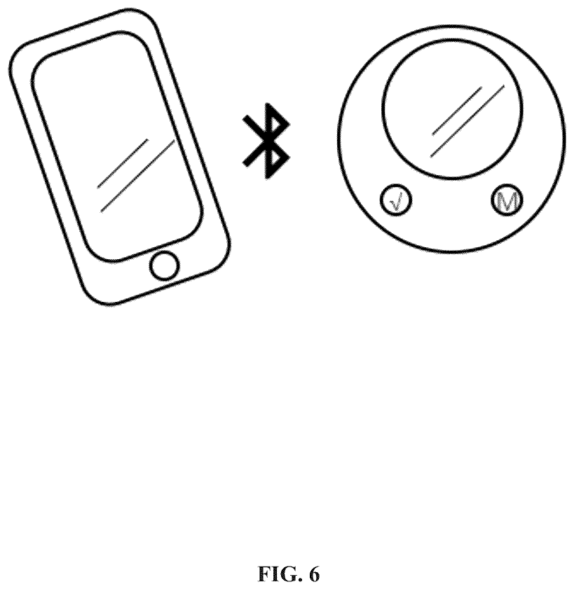

To clearly show the structure of the electronic ruler for pets, FIGS. 2-5 are a left side view and a right side view of the electronic ruler for pets, a front view of the electronic ruler for pets, a front view of the electronic ruler for pets after the front cover and the display are removed, and a front view of the electronic ruler for pets after the front cover/display and the controller are removed. The control keys may be arranged on the front face or side face of the electronic ruler for pets, so that it is convenient for operation and use by a single hand during measurement, and the other hand pulls the tape or reel of the electronic ruler. The tape or reel of the electronic ruler is designed as being automatically contracted and clamped.

The first wireless communication module and/or the second wireless communication module may be a Bluetooth module, a WIFI module, a ZigBee module, an NFC module and/or an Ethernet module.

The pet body index measurement system comprises an electronic scale and an upper computer. The upper computer comprises a memory and a second display. The memory is configured to save the pet body index data. The second display is configured to display the pet body index data and exhibit a setup interface for interaction with the electronic ruler for pets. In addition, the upper computer is configured to calculate and convert the pet body index data and perform wireless communication with the electronic ruler for pets through the first wireless communication module.

When the upper computer is an intelligent terminal device, the first wireless communication module and/or the second wireless communication module is preferably a Bluetooth module or an NFC module. When the upper computer is a server or a PC computer, in addition to the short-range Bluetooth module, WIFI module, NFC module or ZigBee module, the first wireless communication module may also be a remotely connected Ethernet module. The process of the connection and wireless communication of the electronic ruler for pets and the upper computer will be described in the following embodiments by taking Bluetooth as an example. The process of the connection and wireless communication of other modules and the upper computer is also similar to that for Bluetooth. FIG. 6 is a schematic diagram of connecting the electronic ruler for pets to an intelligent terminal device through Bluetooth. When in use, the electronic ruler for pets is in a Bluetooth on state when it is turned on, and the intelligent terminal device turns on Bluetooth and searches for nearby Bluetooth devices, so that the electronic ruler for pets can be found. After verified, the intelligent terminal device is connected to the electronic ruler for pets. Subsequently, the data transmission and control command reception/transmission function can be realized.

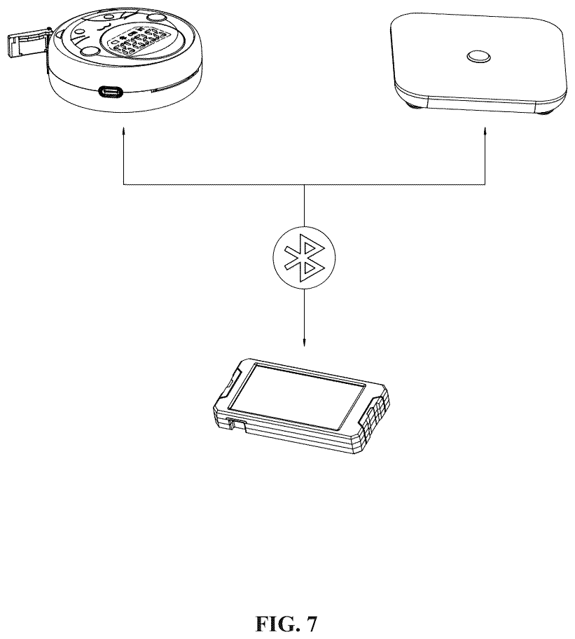

To calculate the body shape parameter of the pet on the intelligent terminal device, the pet body index measurement system further comprises an electronic scale comprising a second wireless communication module. The electronic scale digitally acquires the weight data of the pet, the second wireless communication module is configured to transmit the weight data and receive a control signal transmitted by the upper computer, and the intelligent terminal device can exhibit a setup interface for interaction with the electronic scale. FIG. 7 is a schematic diagram of connecting the electronic ruler for pets and the electronic scale in the pet body index measurement system to an intelligent terminal device through Bluetooth. The step of connecting the electronic scale to the intelligent terminal device through Bluetooth is similar that for the electronic ruler for pets. When the intelligent terminal device does not need to be connected to the electronic ruler for pets and the electronic scale, it is realized in the following way: the Bluetooth is turned off on the intelligent terminal device for temporary disconnection, or the Bluetooth connection device is deleted for permanent disconnection.

If the electronic scale is not used, to obtain the body shape parameter of the pet, another choice is to obtain the weight data of the pet by a general scale and then manually input the weight data into the dialog box of the weight data interface of the intelligent terminal device. The interface described above is embodied as a corresponding application (app) on the intelligent terminal device and embodied as a software program on the server or PC computer.

Here, the pet comprises a dog, a cat or different types, or different varieties in the same type, e.g., Siberian Husky, Labrador, Welsh Corgi, Pomeranian, Golden Retriever, French Bulldog, Poodle, Schnauzer, Chihuahua, Teddy, etc. The type and variety may be selected or filled on the intelligent terminal device.

The process of measuring the neck circumference, chest circumference, body length and body weight of the pet and transmitting and recording the measured data by the pet body index measurement system will be described below. By taking the pet being a dog as an example, the upper computer is an intelligent terminal device.

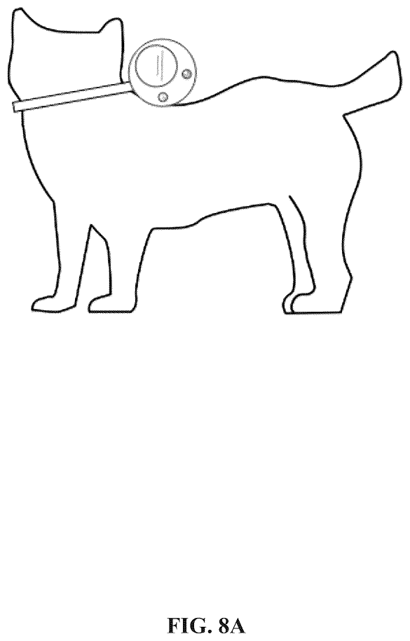

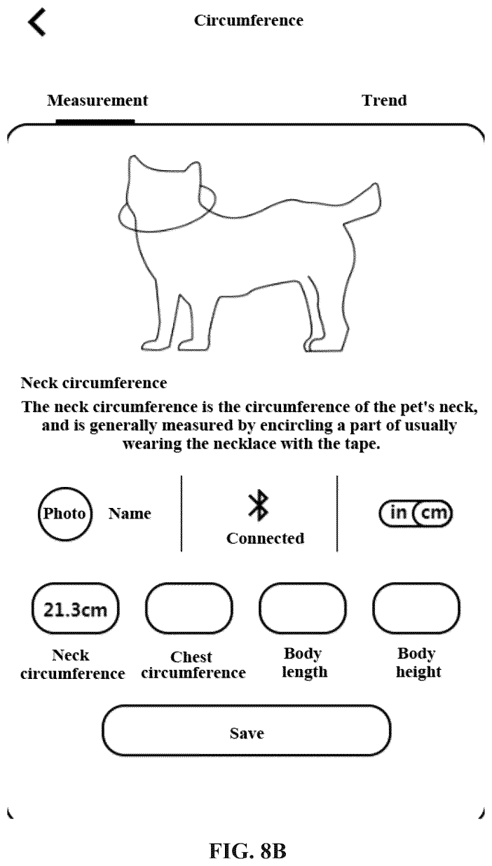

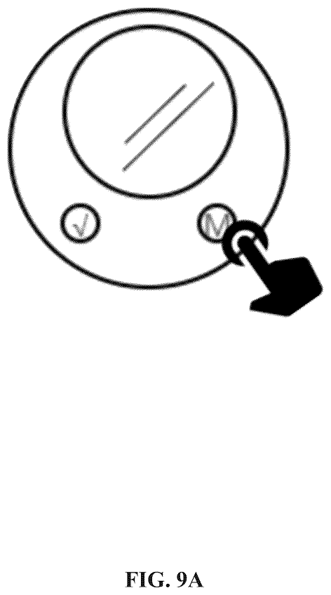

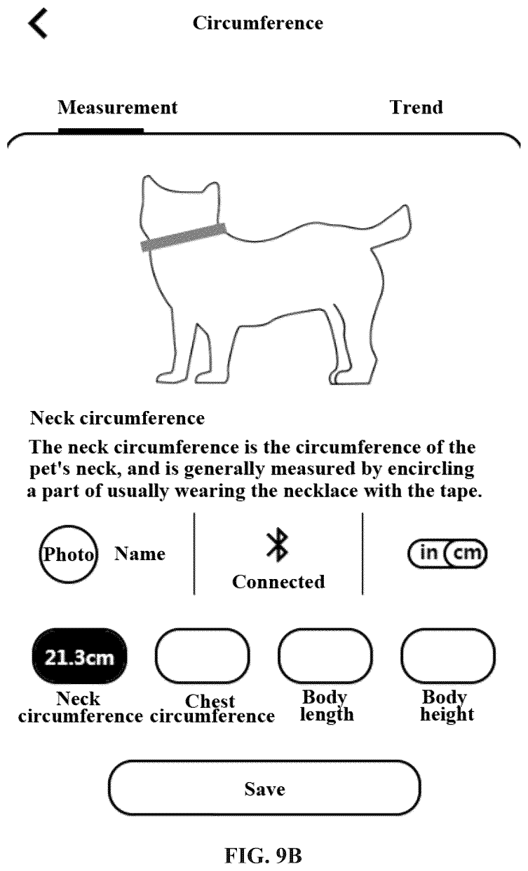

FIGS. 8A-8B and 9A-9B are schematic diagrams of data synchronization and data confirmation of the electronic ruler for pets and the intelligent terminal device during neck circumference measurement. Before measurement, the measurement interface of the app on the intelligent terminal device is opened, comprising neck circumference measurement, and a part for neck circumference measurement will be prompted to provide guidelines for correct measurement, as shown in FIG. 8A. In accordance with the measurement guidelines, the tape or reel of the electronic ruler for pets is pulled out, and the measured length data will be synchronously displayed at the corresponding position of the intelligent terminal device, as shown in FIG. 8B. At this time, the measured data is not confirmed and can be deleted. After the confirm key in the control keys on the electronic ruler for pets is pressed down, as shown in FIG. 9A, the measured data is confirmed. The length data becomes fixed from variable, and different backgrounds are used to show a difference, as shown in FIG. 9B.

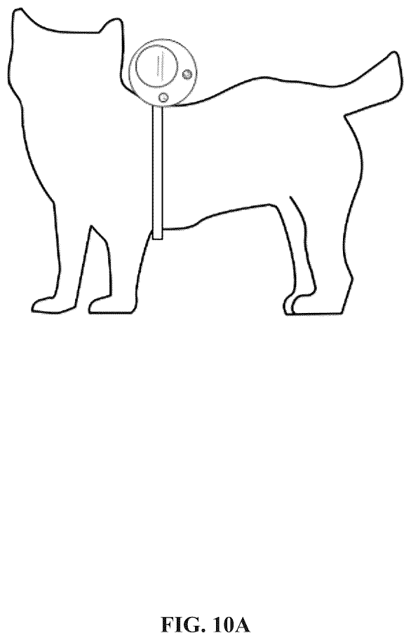

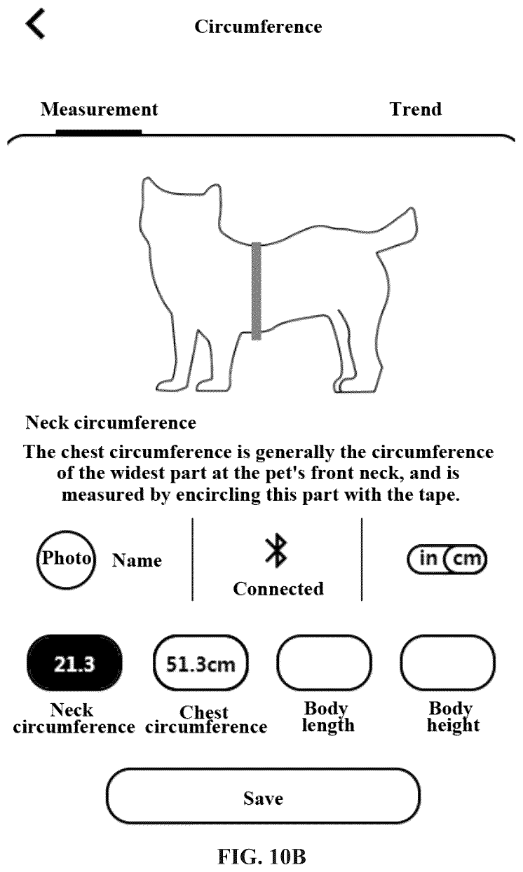

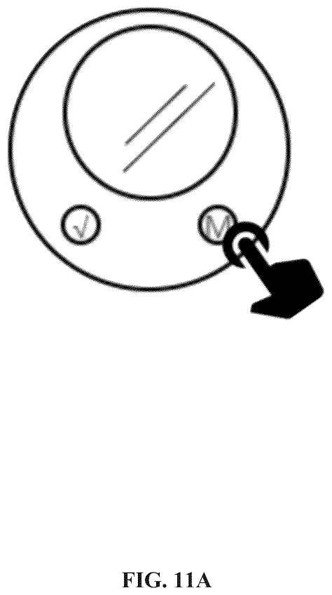

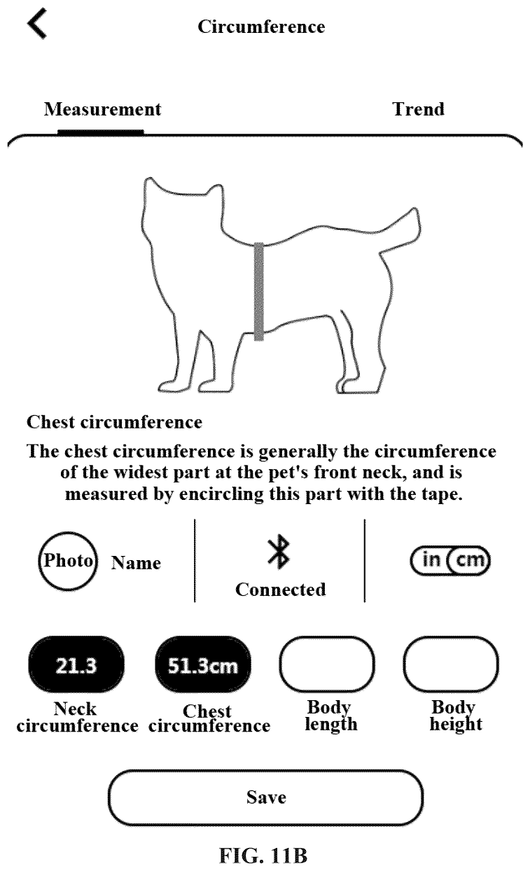

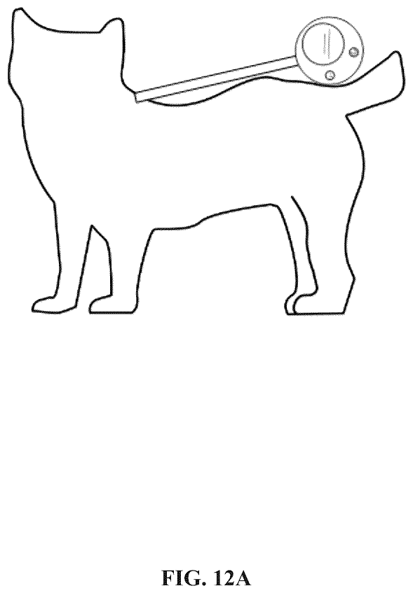

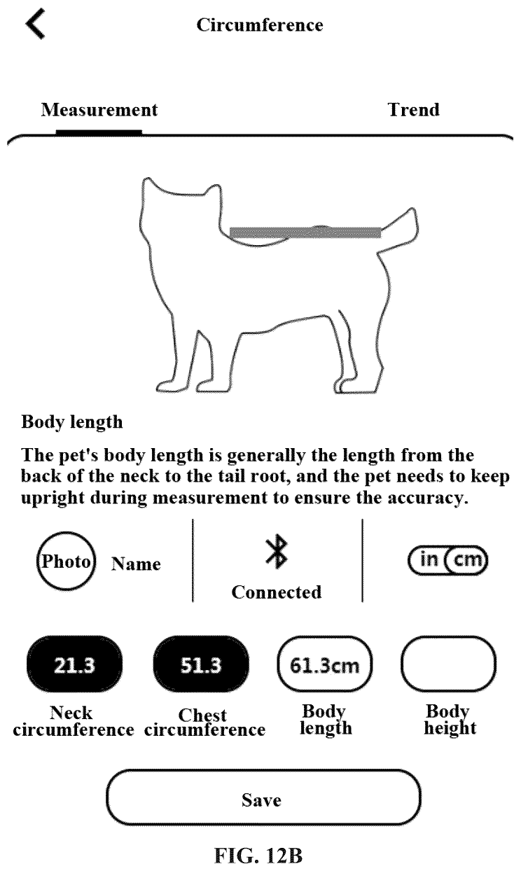

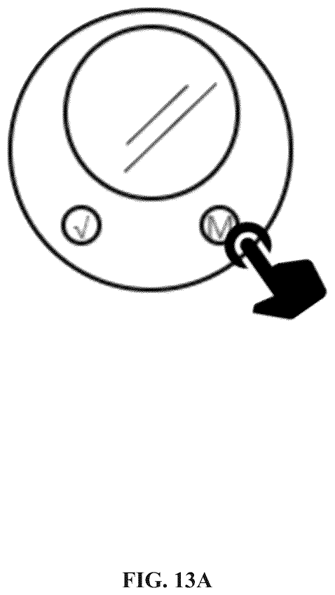

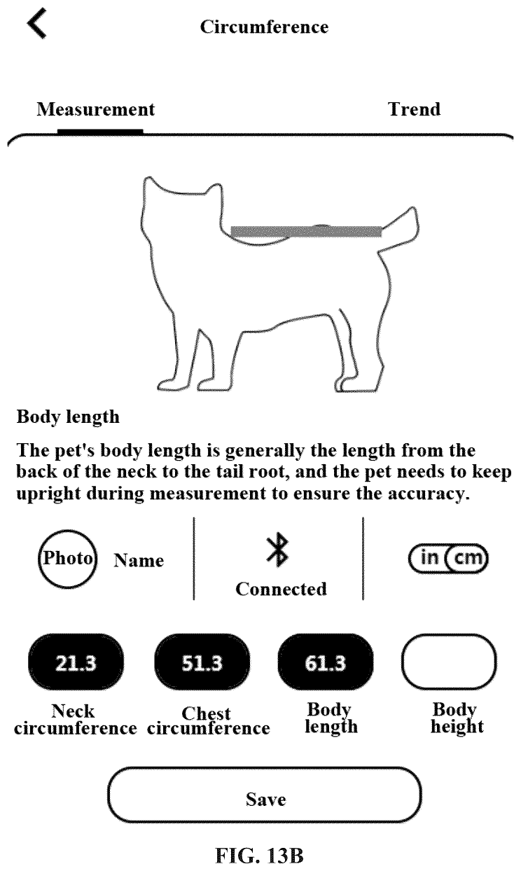

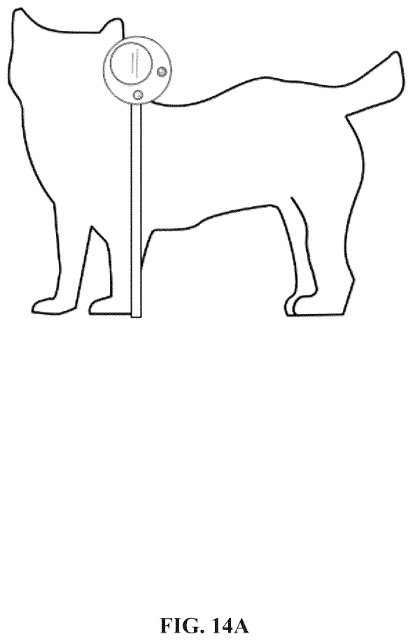

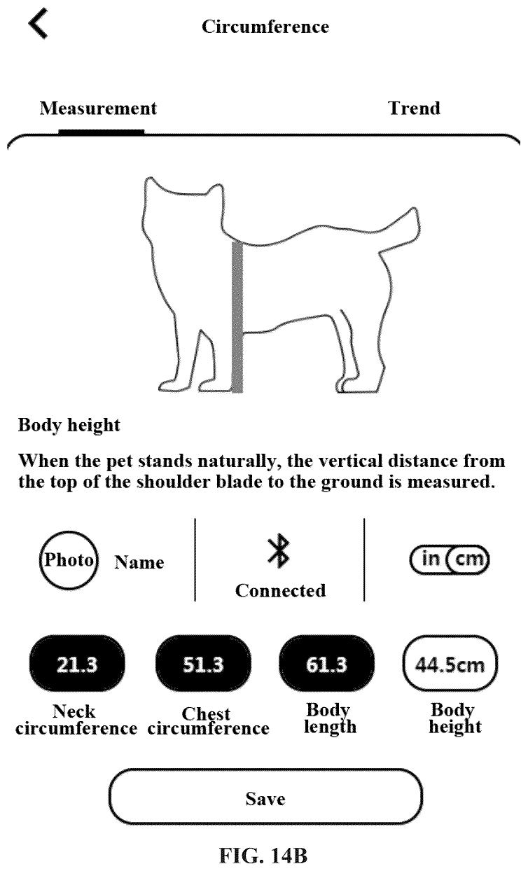

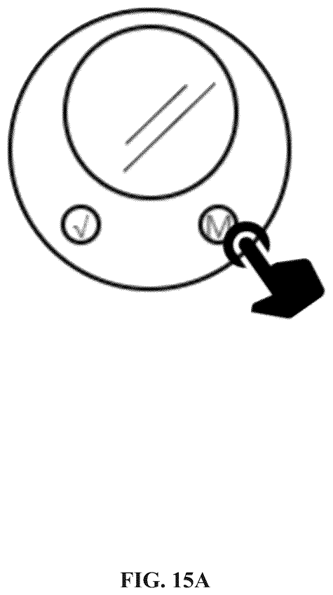

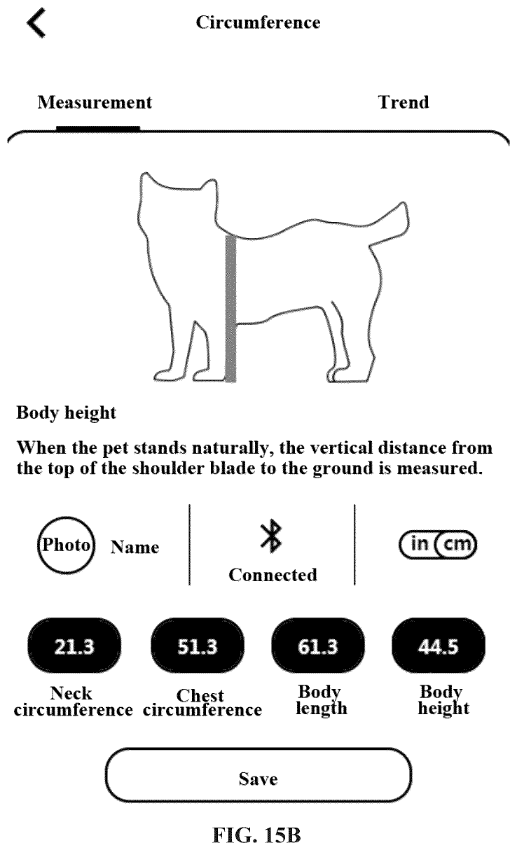

FIGS. 10A-10B and 11A-11B are schematic diagrams of data synchronization and data confirmation of the electronic ruler for pets and the intelligent terminal device during chest circumference measurement. FIGS. 12A-12B and 13A-13B are schematic diagrams of data synchronization and data confirmation of the electronic ruler for pets and the intelligent terminal device during body length measurement. FIGS. 14A-14B and 15A-15B are schematic diagrams of data synchronization and data confirmation of the electronic ruler for pets and the intelligent terminal device during body length measurement.

In addition to the length, length options of other parts, e.g., leg length, tail length, claw width, mouth circumference, leg circumference, etc., may also be added on the measurement interface of the intelligent terminal device. The process of measurement and data recording is similar. When the length data of some parts cannot be measured well at one time or when measurement needs to be performed for multiple times since the pet is hyperactive, the average/sum/difference of the measured data may be taken by the measurement interface.

During weight measurement, the average/sum/difference of the measured data may also be taken. For example, if the weight data of the pet is acquired by a general scale, the weight data when the pet is held a person and the weight data when the pet is not held by the person may be manually input, and the difference between the both is automatically obtained by the app of the intelligent terminal device so as to obtain the weight data of the pet without manual calculation.

The measurement process may be summarized as follows. (1) The electronic ruler for pets or the electronic scale is connected to the intelligent terminal device through the wireless communication module, so that wireless data transmission can be performed after the connection is successful. (2) During measurement, the measurement interface of the intelligent terminal device displays the graph and characters of the length measurement and/or weight measurement of the corresponding body part of the pet to guide the completion of the measurement step, and the measured data on the electronic ruler for pets and/or the electronic scale will be displayed in the corresponding data display box of the intelligent terminal device in real time. (3) The measured data is confirmed and recorded into the application of the intelligent terminal device by the control keys on the intelligent terminal device.

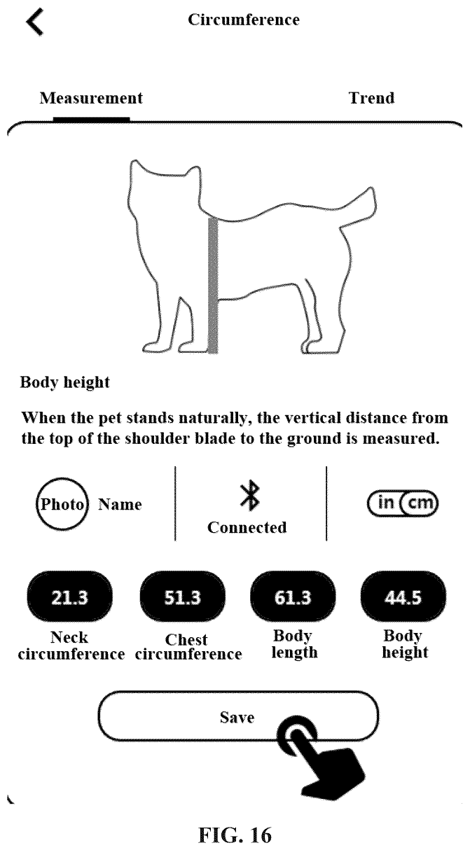

At the end of measurement, SAVE is clicked to save all measured data, as shown in FIG. 16, as the basis for analyzing, processing and applying the pet body index data by the pet body index measurement system. The measured data mainly comprises displaying the growth trend of the pet on the intelligent terminal device, calculating and analyzing the body index of the pet, e.g., BMI and/or BCS scores, etc. These data are also used as the basis for the recommendations of the pet accessories recommendation system.

To expand the recording and guiding function of the pet body index measurement system on the growth of the pet, the upper computer further comprises a camera for shooting a picture of the pet and an algorithm unit for identifying the variety and/or type and the BCS score of the pet according to the picture of the pet, so as to further improve the accuracy of the BMI and/or BCS scores. The BMI parameter range of the heath state of the variety of the bred pet can be analyzed by using a body index measurement database of different pet varieties and/or types, so that the influence of different varieties and/or types of pets on subjective judgment of the body shape is overcome.

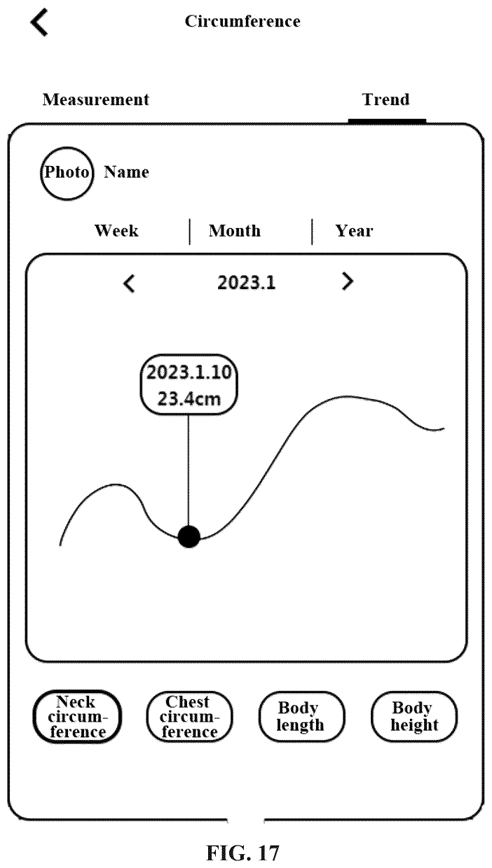

FIG. 17 is a schematic diagram showing the circumference data of the pet in a period of time. The application interface may be set to display the variation trend of the circumference data of each body part of the pet (the time range for viewing the trend can be selected as year/month/week), the historical data of a specific date on the trend curve, and further the record picture.

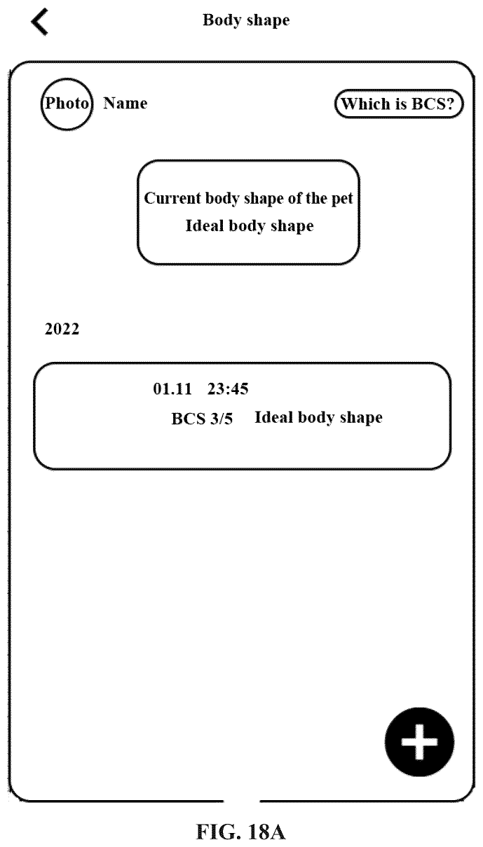

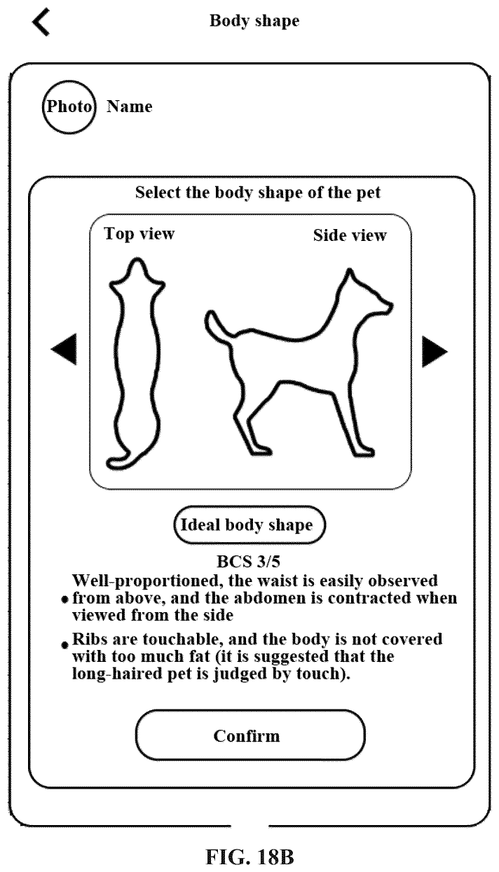

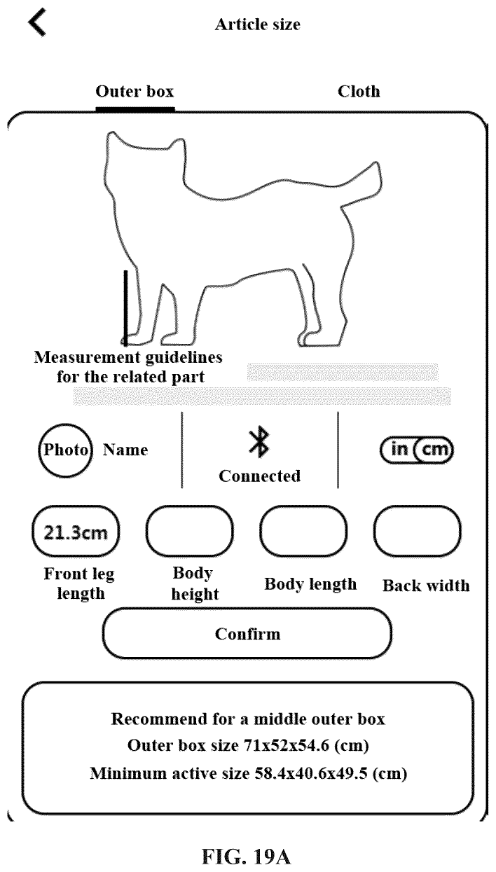

FIGS. 18A-18B are schematic diagrams of the result of body shape analysis of the pet by taking the BCS score as an example. The application interface can display the graph and character guidelines for helping to judge the body shape interval of the pet, so as to guide the user to judge and record the current body shape of the pet, and can also record the body shape of the pet according to the timeline.

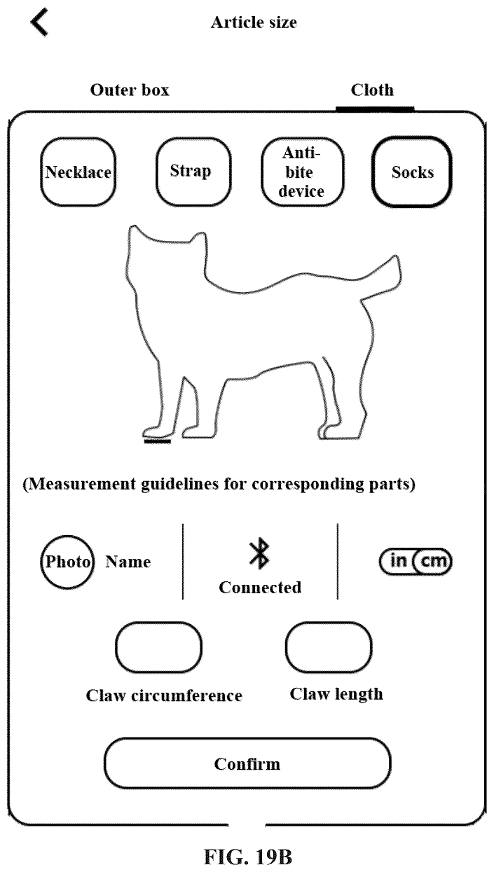

FIGS. 19A-19B are schematic diagrams of recommendations for the space size (FIG. 19A) of the pet box and the cloth size (FIG. 19B). The size of the required IATA pet box is calculated by the following formula: length=back length+½ front leg length; width=back length×2; height=body height+3 inches. For the cloth size recommendation, for example, for the selection of clothing “socks”, the page measurement part is the corresponding “claw length” and “claw width”, and the data after the measurement is completed as shown can be used as reference data in the process of purchasing or making this kind of pet clothes.

It will be obvious to those skilled in the art that changes and modifications may be made, and therefore, the aim in the appended claims is to cover all such changes and modifications.

What is claimed is:

1. An electronic ruler for pets, comprising:
a displacement sensor module;
a controller;
a first display;
a first wireless communication module; and
a control key;
wherein:
the displacement sensor module comprises a tape or reel; the tape or reel is configured to convert a physical length of a pet to be measured into a displacement length through a rotation angle of the tape or reel, and the displacement length is further converted into an electrical signal;
the controller is configured to calculate and convert the electrical signal into a length value in a set unit, and display the length value on the first display; and
the first wireless communication module is configured to transmit the length value to an upper computer, and the control key is configured to set the parameters of the length value and confirm the transmission of the length value to the upper computer.

2. The electronic ruler of claim 1, wherein the displacement sensor module is an absolute capacitive grating sensor or an optical grating sensor.

3. The electronic ruler of claim 1, wherein the first wireless communication module is a Bluetooth module, a WIFI module, a ZigBee module, an NFC module and/or an Ethernet module.

4. The electronic ruler of claim 1, wherein to set the parameters of the length value comprises one or more of the following: setting a unit of the length value, setting or selecting a position where the length value belongs to the pet, selecting to delete or reserve the length value, selecting an average/sum/difference of several length values, and setting or selecting the length value as an absolute length or a relative length.

5. A pet body index measurement system, comprising the electronic ruler for pets of claim 1 and an upper computer,
wherein:
the upper computer comprises a memory and a second display; the memory is configured to save the pet body index data; the second display is configured to display the pet body index data and exhibit a setup interface for interaction with the electronic ruler for pets;
the upper computer is configured to calculate and convert the pet body index data and perform wireless communication with the electronic ruler for pets through the first wireless communication module; and
the electronic ruler for pets digitally acquires the length data of a body part of the pet, and the first wireless communication module is configured to transmit the length data and receive a control signal transmitted by the upper computer.

6. The system of claim 5, wherein
the measurement system further comprises an electronic scale comprising a second wireless communication module; and the upper computer is configured to calculate and convert the pet body index data, perform wireless communication with the electronic ruler through the first wireless communication module and/or perform wireless communication with the electronic ruler through the second wireless communication module;
the electronic ruler digitally acquires the length data of the body part of the pet, and the first wireless communication module is configured to transmit the length data and receive the control signal transmitted by the upper computer; the electronic scale digitally acquires weight data of the pet, and the second wireless communication module is configured to transmit the weight data and receive the control signal transmitted by the upper computer; and
the upper computer calculates BMI and BCS scores for the pet according to the variety of the pet, the length data and the weight data.

7. The system of claim 5, wherein the electronic rule comprises keys for numbering and confirming the measured length data before the first wireless communication module transmits the length data.

8. The system of claim 5, wherein the length data comprises one or more length data of neck circumference, chest circumference, body length, body height, leg length, tail length, claw width, mouth circumference, and leg circumference.

9. The system of claim 5, wherein information received by the first wireless communication module comprises a measurement time of the length data, and information received by the second wireless communication module comprises a measurement time of the weight data; the length data displayed on the display comprises: under a length index, the horizontal axis is a set time range interval, and the vertical axis is a connection graph of the length data in the time range interval; and, the weight data displayed on the display comprises: under a weight index, the horizontal axis is a set time range interval, and the vertical axis is a connection graph of the weight data in the time range interval, or a length and/or weight display graph of the pet at a single specified time from different perspectives.

10. The system of claim 5, wherein the control signal comprises setting a numbering of the measured length data, a unit and a transmission mode of the length data.

11. The system of claim 5, wherein the upper computer is an intelligent terminal device, a server or a PC computer.

12. The system of claim 5, wherein the upper computer comprises an interface for selecting the variety and/or type of the pet, or a camera for shooting a picture of the pet and an algorithm unit for identifying the variety and/or type of the pet according to a picture of the pet.

13. A pet accessories recommendation system, comprising the pet body index measurement system of claim 5, wherein the recommendation system is configured to complete one or more of the following pet accessories recommendations according to the length data and/or the weight data: calculating a space size of an outer box required by the pet, calculating a size of the pet's clothes or shoes and socks, and calculating a caloric value and/or food intake and/or amount of exercise required by the pet per day.
